(19) United States
(12) Patent Application Publication
(10) Pub. No.: US 2025/0268624 A1
(43) Pub. Date: Aug. 28, 2025

(54) SEAL ASSEMBLIES FOR SURGICAL ACCESS ASSEMBLIES

(71) Applicant: Covidien LP, Mansfield, MA (US)

(72) Inventors: Roy J. PILLETERE, North Haven, CT (US); Jason T. MICKUS, Avon, CT (US); Nicolette R. LAPIERRE, Windsor Locks, CT (US); Matthew A. DININO, Newington, CT (US); Eric L. BROWN, Madison, CT (US)

(73) Assignee: Covidien LP, Mansfield, MA (US)

(21) Appl. No.: 19/204,555

(22) Filed: May 11, 2025

Related U.S. Application Data
(63) Continuation of application No. 16/774,206, filed on Jan. 28, 2020, now Pat. No. 12,324,606.

(51) Int. Cl. A61B 17/34; A61B 17/32

(52) U.S. Cl. CPC A61B 17/3462; A61B 17/3423; A61B 17/3498; A61B 2017/320044

(57) ABSTRACT

Access assemblies include an instrument valve housing and a valve assembly disposed within the cavity of the instrument valve housing. The valve assembly includes a guard assembly, and a seal assembly disposed distal of the guard assembly. The seal assembly includes a support member including a ring portion and a seal portion disposed within the ring portion. The seal assembly further includes a plurality of seal sections extending from the ring portion of the support member. The support member and the plurality of seal sections are integrally formed.

### CROSS-REFERENCE TO RELATED APPLICATION(S)

This application is a continuation of U.S. patent application Ser. No. 16/774,206, filed Jan. 28, 2020, the entire disclosure of which is incorporated herein by reference in its entirety.

### FIELD

The present disclosure relates to surgical access assemblies for minimally invasive surgery. More particularly, the present disclosure relates to seal assemblies for surgical access assemblies.

### BACKGROUND

In order to facilitate minimally invasive surgery, a working space must be created at the desired surgical site. An insufflation fluid, typically CO₂, is introduced into the abdomen of the patient to create an inflated state called a pneumoperitoneum. Access assemblies are utilized to allow the introduction of surgical instrumentation and endoscopes (or other visualization tools). These access assemblies maintain the pressure for the pneumoperitoneum, as they have one or more seals that adapt to the surgical instrumentation. Typically, a “zero-seal” in the access assembly seals the access assembly in the absence of a surgical instrument in the access assembly, and an instrument seal seals around a surgical instrument that has been inserted through the access assembly.

The breadth of surgical instrumentation on the market today requires a robust seal capable of adjusting to multiple sizes and withstanding multiple insertions of surgical instrumentation. Some of the instrumentation can include sharp edges that can tear or otherwise damage seals. Therefore, it would be beneficial to have an access assembly with improved seal durability.

### SUMMARY

An access assembly includes an instrument valve housing including upper, lower, and inner housing sections and defining a cavity. A valve assembly is disposed within the cavity of the instrument valve housing. The valve assembly includes a guard assembly, and a seal assembly disposed distally of the guard assembly. The seal assembly includes a support member and a plurality of seal sections connected to the support member. The support member includes a ring portion and a seal portion disposed within the ring portion. The support member and the plurality of seal sections are integrally formed.

In embodiments, the seal assembly includes first, second, third, fourth seal, fifth, and sixth seal sections. The ring portion may be hexagonal. Each seal section of the plurality of seal sections may include a substantially wing shape. An inner edge of each seal section of the plurality of seal sections may be tapered.

In some embodiments, each seal section of the plurality of seal sections is connected to the ring portion by a living hinge. Each seal section of the plurality of seal sections may overlap two adjacent seal sections of the plurality of seal sections in a clockwise direction. Each seal section of the plurality of seal sections may overlap two adjacent seal sections of the plurality of seal sections in a counter-clockwise direction.

The access assembly may further include a retainer assembly including an upper retainer member, a lower retainer member, and a plurality of pins extending from one of the upper or lower retainer members. Each pin of the plurality of pins may be received through an opening in three seal sections of the plurality seal sections and through an opening in the ring portion of the support member.

Also provided is a valve assembly including a guard assembly, and a seal assembly disposed distal of the guard assembly. The seal assembly includes a support member and a plurality of seal sections connected to the support member. The support member includes a ring portion and a seal portion disposed within the ring portion. The support member and the plurality of seal sections are integrally formed.

In embodiments, the seal assembly includes first, second, third, fourth seal, fifth, and sixth seal sections. The ring portion may be hexagonal. Each seal section of the plurality of seal sections may include a substantially wing shape. An inner edge of each seal section of the plurality of seal sections may be tapered.

In some embodiments, each seal section of the plurality of seal sections is connected to the ring portion by a living hinge. Each seal section of the plurality of seal sections may overlap two adjacent seal sections of the plurality of seal sections in a clockwise direction. Each seal section of the plurality of seal sections may overlap two adjacent seal sections of the plurality of seal sections in a counter-clockwise direction.

The access assembly may further include a retainer assembly including an upper retainer member, a lower retainer member, and a plurality of pins extending from one of the upper or lower retainer members. Each pin of the plurality of pins may be received through an opening in three seal sections of the plurality seal sections and through an opening in the ring portion of the support member.

In addition, provided is a seal assembly including a support member having a ring portion and a seal portion disposed within the ring portion, and a plurality of seal sections extending from the ring portion of the support member. The support member and the plurality of seal sections are integrally formed.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiments given below, serve to explain the principles of the disclosure, wherein:

FIG. 1 is a side perspective view of a surgical access assembly according to an embodiment of the present disclosure;

FIG. 2 a side cross-sectional view of the surgical access assembly shown in FIG. 1 taken along section line 2-2;

FIG. 3 is an exploded perspective view of a valve assembly, with parts separated, including a centering mechanism, a guard assembly, a seal assembly, and a retainer assembly;

FIG. 4 is a top perspective view of the seal assembly shown in FIG. 3, in an initial or unfolded condition;

FIG. 5 is a top plan view of the seal assembly shown in FIG. 4, in the initial or unfolded condition;

FIG. 6 is a top perspective view of the seal assembly shown in FIGS. 3 and 4, in a partially folded condition;

FIG. 7 is a top perspective view of the seal assembly shown in FIGS. 4-6, in a fully folded condition;

FIG. 8 is a bottom perspective view of the seal assembly shown in FIG. 7;

FIG. 9 is a bottom perspective view of the valve assembly shown in FIG. 3, as assembled;

FIG. 10 is a top perspective view of a seal assembly according to another aspect of the disclosure, in an initial or unfolded condition;

FIG. 11 is a top plan view of the seal assembly shown in FIG. 10, in the initial or unfolded condition;

FIG. 12 is an enlarged view of the indicated area of detail shown in FIG. 11;

FIG. 13 is top perspective view of the seal assembly shown in FIGS. 10 and 11, in a partially folded condition; and

FIG. 14 is a top perspective view of the seal assembly shown in FIGS. 10-13, in a fully folded condition.

### DETAILED DESCRIPTION

Particular embodiments of the present disclosure are described hereinbelow with reference to the accompanying drawings; however, it is to be understood that the disclosed embodiments are merely exemplary of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure. Like reference numerals refer to similar or identical elements throughout the description of the figures.

As used herein, the term “distal” refers to that portion of the instrument, or component thereof which is farther from the user while the term “proximal” refers to that portion of the instrument or component thereof which is closer to the user. As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

Surgical access assemblies with obturators are employed during minimally invasive surgery, e.g., laparoscopic surgery, and provide for the sealed access of surgical instruments into an insufflated body cavity, such as the abdominal cavity. The surgical access assemblies of the present disclosure include an instrument valve housing mounted on a cannula tube, and include an obturator (not shown) inserted through the valve housing and cannula. The obturator can have a blunt distal end, or a bladed or non-bladed penetrating distal end and can be used to incise the abdominal wall so that the surgical access assembly can be introduced into the abdomen. The handle of the obturator can engage or selectively lock into the instrument valve housing of the surgical access assembly.

Surgical access assemblies are employed to tunnel through an anatomical structure, e.g., the abdominal wall, either by making a new passage through the anatomical structure or by passing through an existing opening through the anatomical structure. Once the surgical access assembly with the obturator has tunneled through the anatomical structure, the obturator is removed, leaving the surgical access assembly in place. The instrument valve housing of the surgical access assembly includes valves that prevent the escape of insufflation fluid from the body cavity, while also allowing surgical instruments to be inserted into the body cavity.

In various embodiments, a bladeless optical trocar obturator may be provided that permits separation of tissue planes in a surgical procedure and visualization of body tissue fibers as they are being separated, thereby permitting a controlled traversal across a body wall. In other embodiments, the trocar obturator may be bladeless without being optical, e.g., without providing contemporaneous visualization thereof through the distal tip of an obturator. The bladeless obturator may be provided for the blunt dissection of the abdominal lining during a surgical procedure.

Various trocar obturators suitable for use with the surgical access assembly of the present disclosure are known and include, for example, bladed, bladeless, blunt, optical, and non-optical. For a detailed description of the structure and function of exemplary trocar assemblies, including exemplar trocar obturators and exemplar cannulas, please refer to PCT Publication No. WO 2016/186905 (“the '905 publication”), the content of which is hereby incorporated by reference herein in its entirety.

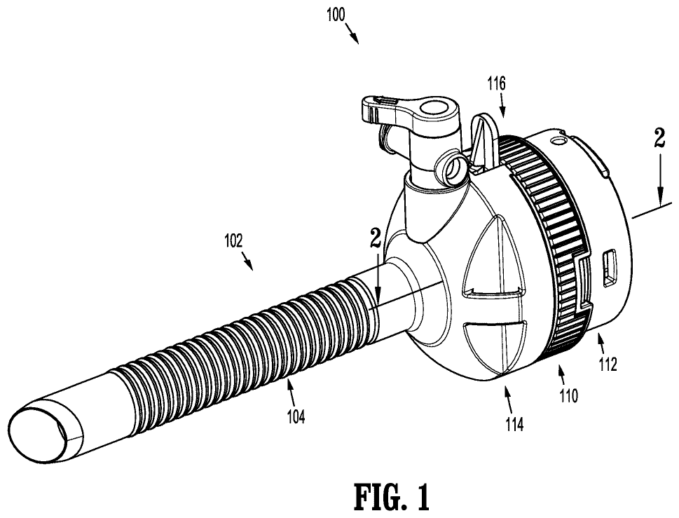

With initial reference now to FIG. 1, a surgical access assembly according to aspects of the present disclosure is shown generally as surgical access assembly 100. The surgical access assembly 100 includes a cannula 102 and an instrument valve housing 110 secured to the cannula 102. For a detailed description of an exemplary surgical access assembly, please refer to the '905 publication.

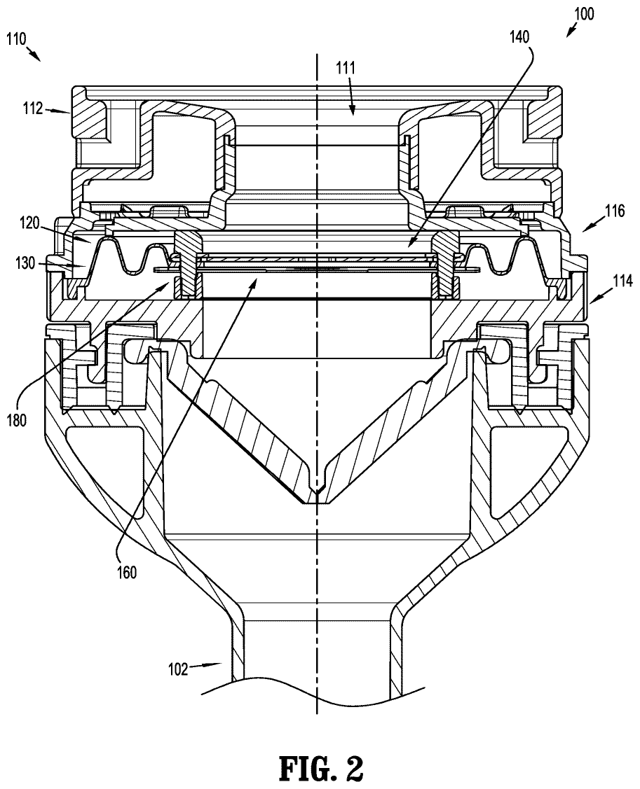

With reference to FIG. 2, the instrument valve housing 110 of the surgical access assembly 100 includes an upper housing section 112, a lower housing section 114, and an inner housing section 116. The upper, lower, and inner housing sections 112, 114, 116 are configured to support a valve assembly 120 on a proximal end of the cannula 102. More particularly, the inner housing section 116 is secured between the upper and lower housing sections 112, 114, and the valve assembly 120 is received between the inner and lower housing sections 116, 114. The upper and lower housing sections 112, 114 of the instrument valve housing 110 may be selectively attachable to, and detachable from, the inner housing section 116. The lower housing section 114 may be releasably or permanently attached to a cannula tube 104 (FIG. 1) of the cannula 102. In embodiments, either or both of the upper and lower housing sections 112, 114 of the instrument valve housing 110 may include knurls, indentations, tabs, or be otherwise configured to facilitate engagement by a clinician.

The surgical access assembly 100 may also include features for the stabilization of the surgical access assembly. For example, the distal end of the cannula tube 104 may carry a balloon anchor or another expandable member that engages the abdomen from the interior side. For example, see U.S. Pat. No. 7,300,448, the entire disclosure of which is hereby incorporated by reference herein. A feature on the opposite side of the abdominal wall may be used to further stabilize the surgical access assembly, such as adhesive tabs or adjustable foam collars.

The upper, lower, and inner housing sections 112, 114, 116 of the instrument valve housing 110 define a longitudinal passage 111 for receipt of a surgical instrument (not shown). The valve assembly 120 is supported within the instrument valve housing 110 to provide sealed passage of the surgical instrument (not shown) through the surgical access assembly 100.

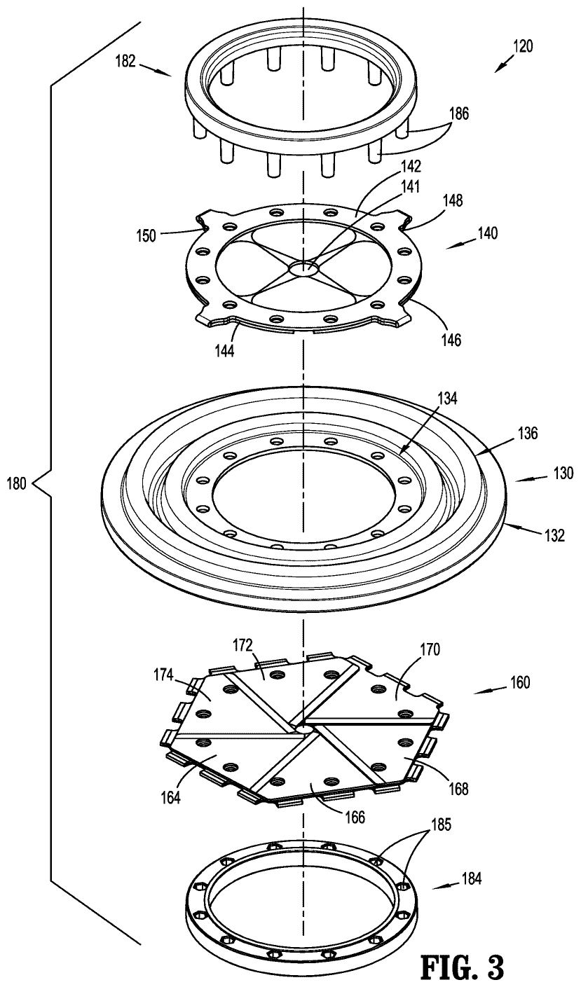

With particular reference to FIGS. 2 and 3, the valve assembly 120 supported in the instrument valve housing 110 (FIG. 2) includes a centering mechanism 130, a guard assembly 140, a seal assembly 160, and a retainer assembly 180. The centering mechanism 130 of the valve assembly 120 permits radial movement of the valve assembly 120 relative to the instrument valve housing 110 when a surgical instrument is received through the valve assembly 120, and returns the valve assembly 120 to a generally centered position once the surgical instrument is withdrawn from within the instrument valve housing 110. The guard assembly 140 protects the seal assembly 160 during insertion and withdrawal of a surgical instrument through the seal assembly 160. The seal assembly 160 provides scaled passage of the surgical instrument through the instrument valve housing 110. The retainer assembly 180 maintains the centering mechanism 130, the guard assembly 140, and the seal assembly 160 in an aligned relationship with one another.

With continued reference to FIGS. 2 and 3, as noted above, the centering mechanism 130 of the valve assembly 120 is configured to maintain the valve assembly 120 centered within the instrument valve housing 110 (FIG. 2) in the absence of a surgical instrument passing through the valve assembly 120. In embodiments, and as shown, the centering mechanism 130 includes an outer annular ring 132, an inner annular ring 134, and a bellows 136 disposed between the outer annular ring 132 and the inner annular ring 134. As shown in FIG. 2, the outer annular ring 132 is received between the inner housing section 116 and the lower housing section 114 to retain the centering mechanism 130 within the instrument valve housing 110. The inner annular ring 134 supports the guard assembly 140. For a detailed description of the structure and function of an exemplary centering mechanism, please refer to U.S. Pat. No. 6,702,787, the content of which is incorporated herein by reference in its entirety.

Although shown including the centering mechanism 130 having bellows 136, the valve assembly 120 may include alternative centering mechanisms. For example, the centering mechanism may include an annular base and a plurality of spokes extending from the base, as described in U.S. Pat. App. Pub. No. 2015/0025477 (“the '477 publication”), the content of which is incorporated herein by reference in its entirety. It is envisioned that the centering mechanism may include multiple sets of spokes, as disclosed in the '477 publication.

With continued reference to FIGS. 2 and 3, the guard assembly 140 of the valve assembly 120 is configured to protect the seal assembly 160 as a surgical instrument (not shown) passes through the instrument valve housing 110 (FIG. 2). The guard assembly 140 includes a ring portion 142 and first, second, third, and fourth petals 144, 146, 148, 150. The first, second, third, and fourth petals 144, 146, 148, 150 define an opening 141 therebetween to facilitate sealed passage of a surgical instrument (not shown) through the guard assembly 140. Although shown including four (4) petals, it is envisioned that the guard assembly may include any suitable number of petals, and the petals may include flap portions of any size or configuration. For detailed description of the structure and function of other exemplary guard assemblies, please refer to U.S. Pat. Nos. 5,895,377 and 6,569,120 (“the '377 and '120 patents”) and U.S. patent application Ser. Nos. 16/394,043 and 16/238,823, the entire disclosures of which are incorporated herein by reference in their entirety.

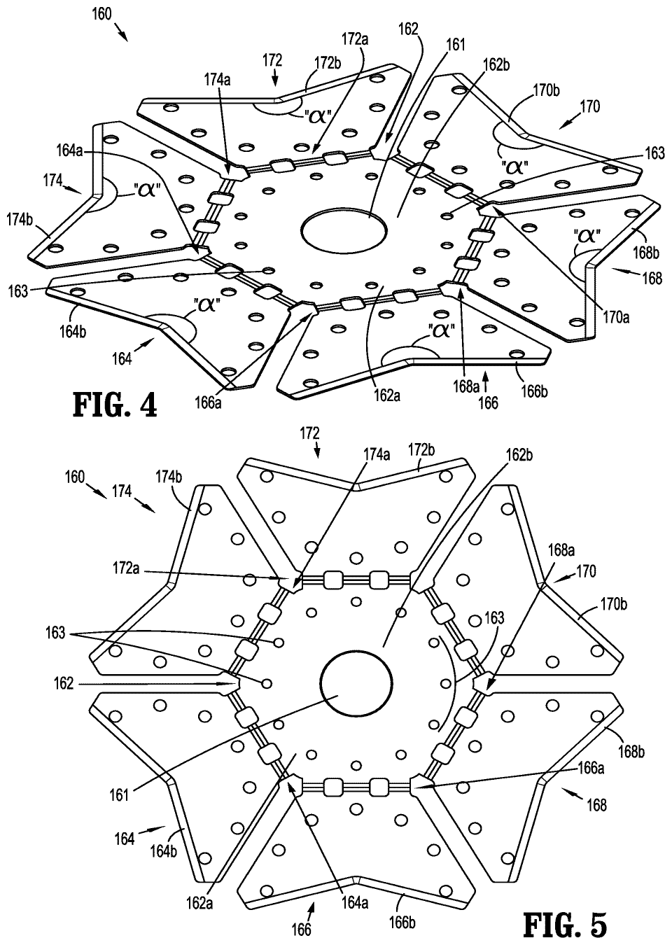

Referring to FIGS. 4-6, the seal assembly 160 of the valve assembly 120 is configured to provide a seal around an outer surface of a surgical instrument passing through the instrument valve housing 110 (FIG. 1). In embodiments, and as shown, the seal assembly 160 forms a flat seal body; however, it is envisioned that the aspects of the present disclosure may be modified for use with a conical seal body.

The seal assembly 160 includes a support member 162, and first, second, third, fourth, fifth, and sixth seal sections 164, 166, 168, 170, 172, 174 secured to the support member 162 by respective connector portions 164a, 166a, 168a, 170a, 172a, 174a. The connector portions 164a, 166a, 168a, 170a, 172a, 174a may include one or more living hinges, as shown, or be otherwise configured to permit folding of the respective first, second, third, fourth, fifth, and sixth seal sections 164, 166, 168, 170, 172, 174 relative to the support member 162.

In embodiments, the support member 162 and the first, second, third, fourth, fifth, and sixth seal sections 164, 166, 168, 170, 172, 174 of the seal assembly 160 are formed of the same material, including, for example, polyurethane, polyisoprenes, or silicone elastomers. Alternatively, the support member 162, and the first, second, third, fourth, fifth, and sixth seal sections 164, 166, 168, 170, 172, 174 may be formed of different materials. In embodiments, the support member 162 and/or the first, second, third, fourth, fifth, and sixth seal sections 164, 166, 168, 170, 172, 174 may include one or more fabric layers.

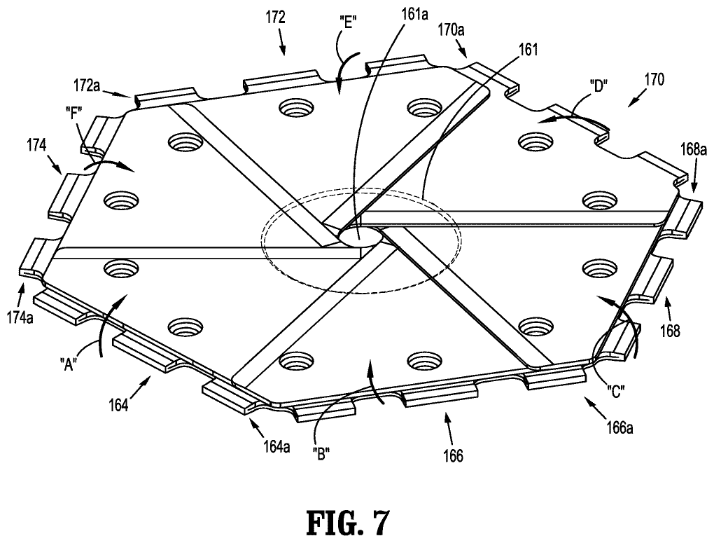

As noted above, the first, second, third, fourth, fifth, and sixth seal sections 164, 166, 168, 170, 172, 174 of the seal assembly 160 define a planar configuration when the seal assembly 160 is in the folded condition (FIG. 7). However, the seal assembly 160 may define a conical seal.

The support member 162 of the seal assembly 160 includes a hexagonal body or ring portion 162a and a seal portion 162b supported within the support member 162a. The ring portion 162a and the seal portions 162b may be formed of the same or different materials. The seal portion 162b defines a central opening 161. The support member 162a defines a plurality of openings 163 corresponding to a plurality of pins 186 (FIG. 3) extending from an upper retainer member 182 of the retainer assembly 180. The seal portion 162a provides additional support to the first, second, third, fourth, fifth, and sixth seal sections 164, 166, 168, 170, 172, 174 when the seal assembly 160 is in the folded configuration.

In embodiments, and as shown, the ring portion 162a and the seal portion 162b of the support member 162 are of unitary construction, i.e., monolithic, with the first, second, third, fourth, fifth, and sixth seal sections 164, 166, 168, 170, 172, 174. By incorporating the ring portion 162a with the seal portion 162b of the support member 162 of the seal assembly 160 and forming the first, second, third, fourth, fifth, and sixth seal sections 164, 166, 168, 170, 172, 174 on the ring portion 162a of the support member 162, instead of including a ring portion for the seal portion and a separate ring portion for the first, second, third, fourth, fifth, and sixth seal sections 164, 166, 168, 170, 172, 174, the number of components in the seal assembly 160, and ultimately, the valve assembly 120, is reduced. The incorporation of the seal portion 162b and the first, second, third, fourth, fifth, and sixth seal sections 164, 166, 168, 170, 172, 174 with the same ring portion 162 also reduces assembly time and reduces material costs.

When in the folded condition (FIG. 7), the first, second, third, fourth, fifth, and sixth seal sections 164, 166, 168, 170, 172, 174 of the seal assembly 160 define an opening 161a configured to receive a surgical instrument (not shown) inserted through the valve assembly 120 in a sealed manner. The first, second, third, fourth, fifth, and sixth seal sections 164, 166, 168, 170, 172, 174 form a non-continuous or virtual seal circumference to reduce tearing during insertion, manipulation, and/or withdrawal of a surgical instrument (not shown) through the valve assembly 120.

Each of the first, second, third, fourth, fifth, and sixth seal sections 164, 166, 168, 170, 172, 174 of the seal assembly 160 includes a wing-shaped body. When the seal assembly 160 is in the folded configuration, the first, second, third, fourth, fifth, and sixth seal sections 164, 166, 168, 170, 172, 174 are configured to partially overlap two adjacent seal sections in each of the clockwise and the counter-clockwise directions. More particularly, the first seal section 164 is configured to partially overlap, the sixth and fifth seal sections 174, 172 in the clockwise direction and the second and third seal section 166, 168 in the counter-clockwise direction, the second seal section 164 is configured to partially overlap the first and sixth seal sections 164, 174 in the clockwise direction and the third and fourth seal sections 164, 168 in the counter-clockwise direction, the third seal section 166 is configured to partially overlap the second and first seal sections 166, 164 in the clockwise direction and the fourth and fifth seal section 170, 172 in the counter-clockwise direction, the fourth seal section 170 is configured to partially overlap the third and second seal sections 168, 166 in the clockwise direction and the fifth and sixth seal sections 172, 174 in the counter-clockwise direction, the fifth seal section 172 is configured to partially overlap the fourth and third seal sections 170, 168 in the clockwise direction and the sixth and first seal sections 174, 164 in the counter-clockwise direction, and the sixth seal section 174 is configured to partially overlap the fifth and fourth seal sections 172, 170 in the clockwise direction and the first and second seal sections 164, 166 in the counter clockwise direction.

In embodiments, and as shown, the first, second, third, fourth, fifth, and sixth seal sections 164, 166, 168, 170, 172, 174 are folded such that the first, second, third, fourth, fifth, and sixth seal sections 164, 166, 168, 170, 172, 174 overlap in a sequential pattern in counter-clockwise direction. Alternatively, the first, second, third, fourth, fifth, and sixth seal sections 164, 166, 168, 170, 172, 174 may folded to overlap in a sequential clockwise direction. It is envisioned that the first, second, third, fourth, fifth, and sixth seal sections 164, 166, 168, 170, 172, 174 may instead be folded in opposed pairs, i.e., first and fourth seal sections 164, 170 folded together, second and fifth seal sections, 166, 172 folded together, and third and sixth seal sections 168, 174 folded together, or in any other suitable manner.

An inner edge 164b, 166b, 168b, 170b, 172b, 174b of the respective first, second, third, fourth, fifth, and sixth seal sections 164, 166, 168, 170, 172, 174 of the seal assembly 160 may define a V-shape, as shown, or may extend straight across. In embodiments, the V-shape defines an angle “α” from about one-hundred eighty degrees (180°) to about two-hundred seventy-five degrees (275°)(FIG. 4). The V-shape of the inner edges 164b, 166b, 168b, 170b, 172b 164b facilitates reception of a surgical instrument (not shown) through the seal assembly 160.

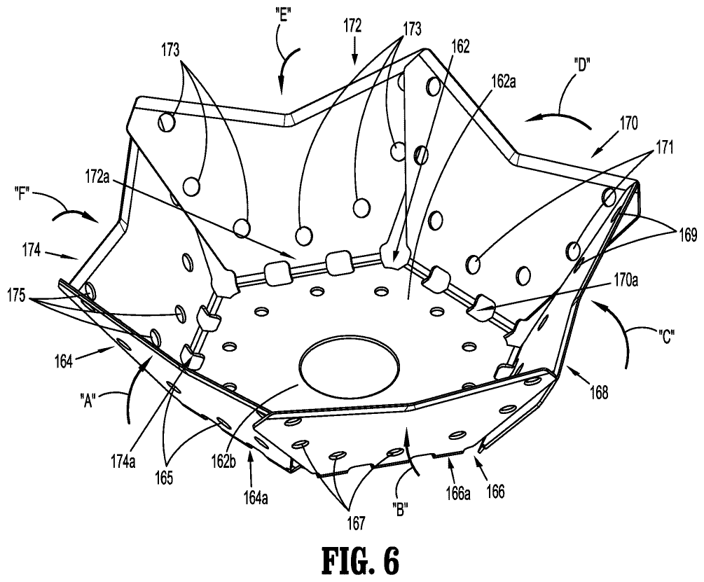

Each of the first, second, third, fourth, fifth, and sixth seal sections 164, 166, 168, 170, 172, 174 defines a plurality of openings 165, 167, 169, 171, 173, 175 (FIG. 6) adjacent the respective connector portions 164a, 166a, 168a, 170a, 172a, 174a of each seal section 164, 166, 168, 170, 172, 174, respectively. In embodiments, and as shown, the plurality of openings 165, 167, 169, 171, 173, 175 are arranged such each opening of the plurality of openings 163 in the support member 162 is aligned with an opening of the plurality of openings 165, 167, 169, 171, 173, 175 of the respective first, second, third, fourth, fifth, and sixth seal sections 164, 166, 168, 170, 172, 174 and an opening of the plurality of openings 165, 167, 169, 171, 173, 175 of the two adjacent first, second, third, fourth, fifth, and sixth seal sections 164, 166, 168, 170, 172, 174. In this manner, each pin of the plurality of pins 186 (FIG. 3) of the retainer assembly 180 is received through an opening 163 in the support member 162 and through an opening 165, 167, 169, 171, 173, 175 in three of the six seal sections 164, 166, 168, 170, 172, 174 when the first, second, third, fourth, fifth, and sixth seal sections 164, 166, 168, 170, 172, 174 are secured relative to each other in the assembled configuration. This arrangement ensures the integrity of the seal assembly 160, and more particularly, ensures the positioning of the first, second, third, fourth, fifth, and sixth seal sections 164, 166, 168, 170, 172, 174 relative to each other and the support member 162.

FIGS. 6 and 7 illustrate the method of folding the seal assembly 160. As shown in FIG. 6, each of the first, second, third, fourth, fifth, and sixth seal sections 164, 166, 168, 170, 172, 174 are folded relative to the support member 162 at the respective connector portion 164a, 166a, 168a, 170a, 172a, 174a, as indicated by arrows “A”, “B”, “C”, “D”, “E”, and “F” respectively. As shown, the first, second, third, fourth, fifth, and sixth seal sections 164, 166, 168, 170, 172, 174 are folded simultaneously, with adjacent seal sections overlapping one another. More particularly, the second seal section 166, overlaps the first seal section 164, the third seal section 168 overlaps the second seal section 166, and so on until the first seal section 164 overlaps the sixth seal section 174. This overlapping or interweaving pattern increases the integrity of the seal assembly 160, i.e., reduces the likelihood of the seal assembly 160 leaking when a surgical instrument is received therethrough. As noted above, alternatively, the seal sections may be folded in any manner, including, with opposed seal sections folded together.

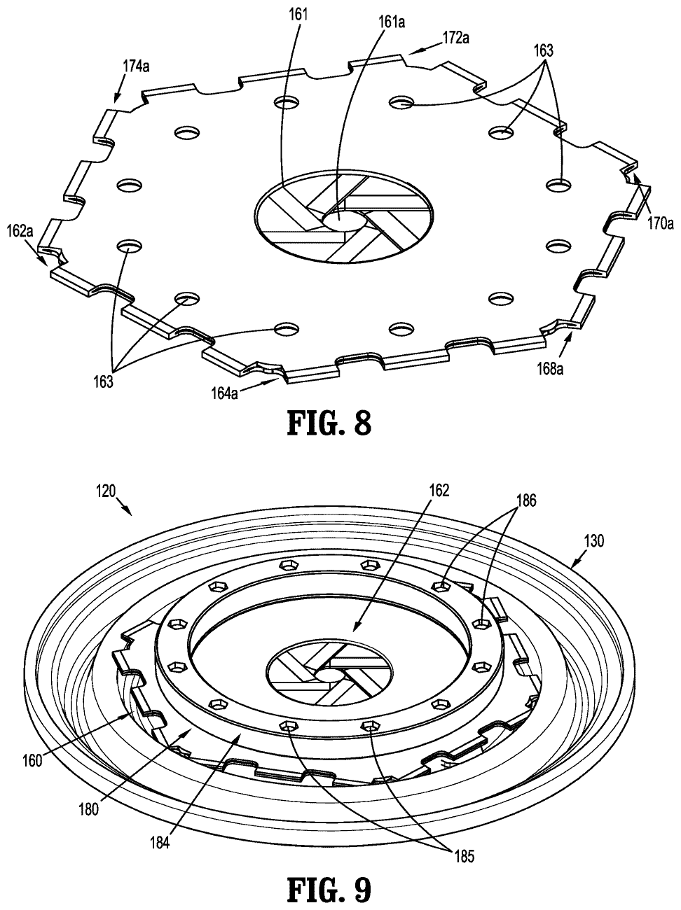

FIGS. 7 and 8 illustrate the seal assembly 160 in the folded condition. In the folded configuration, the first, second, third, fourth, fifth, and sixth seal sections 164, 166, 168, 170, 172, 174 are disposed adjacent the seal portion 162b of the support member 162.

FIG. 9 illustrates a bottom view of the assembled valve assembly 120. The seal assembly 160 is secured relative to the centering mechanism 130 and the guard assembly 140 (FIG. 3) by retainer assembly 180. More particularly, the plurality of pins 186 extending from the upper retainer member 182 (FIG. 3) of the retainer assembly 180 extend through the guard assembly 140, the centering mechanism 130, and the seal assembly 160, and are secured within openings 185 of the lower retainer member 184. In embodiments, the plurality of pins 186 is welded, glued, adhered, bonded or otherwise secured within the plurality of openings 185 in the lower retainer member 184 to secure the upper retainer member 182 and the lower retainer member 184 together. The lower retainer member 184 may instead, or additionally, include a plurality of pins (not shown) with the upper retainer member 182 defining a plurality corresponding openings (not shown). Either or both of the upper and lower retainer members 182, 184 may include locking features (not shown) for engaging the plurality of pins 186 and securing the upper retainer member 182 to the lower retainer member 184.

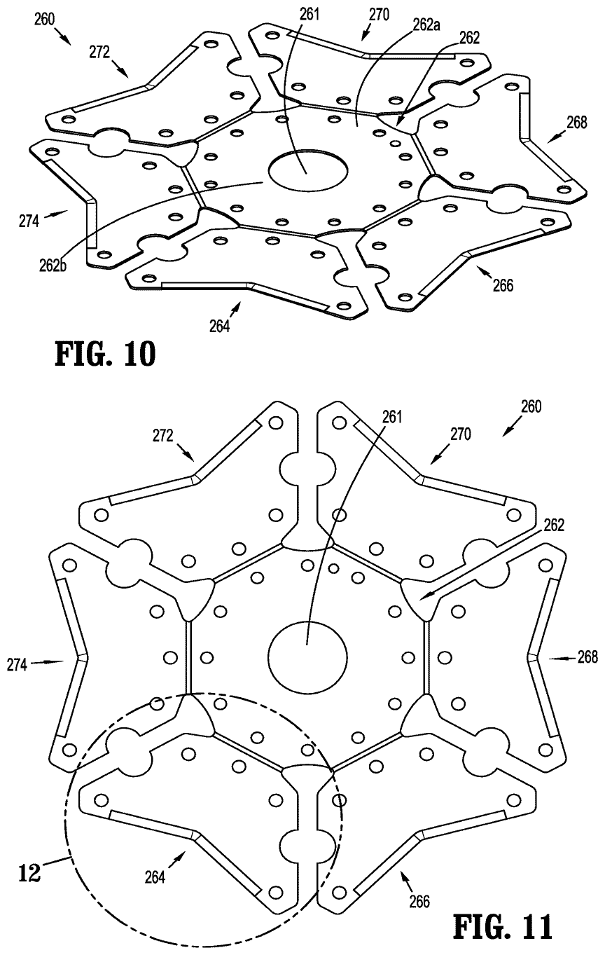

Referring to FIGS. 10-13, a seal assembly according to another aspect of the disclosure is shown generally as seal assembly 260. The seal assembly 260 is substantially similar to the seal assembly 160 described hereinabove, and will only be described in detail with regards to the differences therebetween. The seal assembly 260 is configured to be received within the instrument valve housing 110 (FIG. 1) and provide a seal around an outer surface of a surgical instrument passing through the instrument valve housing 110. As shown, the seal assembly 260 forms a flat seal body; however, it is envisioned that the aspects of the disclosure may be modified for use with a conical seal body.

The seal assembly 260 includes a support member 262, and first, second, third, fourth, fifth, and sixth seal sections 264, 266, 268, 270, 272, 274 secured to the support member 262. In some aspects of the disclosure, the support member 262 and the first, second, third, fourth, fifth, and sixth seal sections 264, 266, 268, 270, 272, 274 of the seal assembly 260 are formed of the same material, including, for example, polyurethane, polyisoprenes, or silicone elastomers. Alternatively, the support member 262, and the first, second, third, fourth, fifth, and sixth seal sections 264, 266, 268, 270, 272, 274 are formed of different materials. In other aspects, the support member 262 and/or the first, second, third, fourth, fifth, and sixth seal sections 264, 266, 268, 270, 272, 274 may include one or more fabric layers.

As noted above, the first, second, third, fourth, fifth, and sixth seal sections 264, 266, 268, 270, 272, 274 of the seal assembly 260 define a planar configuration when the seal assembly 260 is in the folded condition (FIG. 14). However, the seal assembly 260 may define a conical seal when in the folded condition.

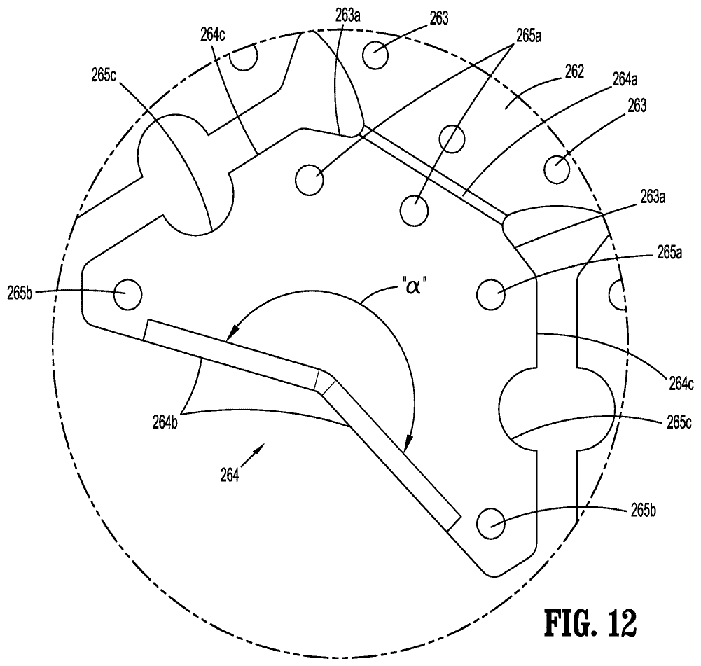

The support member 262 of the seal assembly 260 includes a hexagonal body or ring portion 262a and a seal portion 262b supported within the support member 262a. The ring portion 262a and the seal portions 262b may be formed of the same or different materials. The seal portion 262b defines a central opening 261. The support member 262a defines a plurality of openings 263 (FIG. 12) corresponding to the plurality of pins 186 (FIG. 3) extending from the upper retainer member 182 of the retainer assembly 180. The seal portion 262a provides additional support to the first, second, third, fourth, fifth, and sixth seal sections 264, 266, 268, 270, 272, 274 when the seal assembly 260 is in the folded configuration (FIG. 14).

As shown, the ring portion 262a and the seal portion 262b of the support member 262 are of unitary construction, i.e., monolithic, with the first, second, third, fourth, fifth, and sixth seal sections 264, 266, 268, 270, 272, 274. By incorporating the ring portion 262a with the seal portion 262b of the support member 262 of the seal assembly 260 and forming the first, second, third, fourth, fifth, and sixth seal sections 264, 266, 268, 270, 272, 274 on the ring portion 262a of the support member 262, instead of including a ring portion for the seal portion and a separate ring portion for the first, second, third, fourth, fifth, and sixth seal sections 264, 266, 268, 270, 272, 274, the number of components in the seal assembly 260, and ultimately, the valve assembly 120, is reduced (FIG. 3). The incorporation of the seal portion 262b and the first, second, third, fourth, fifth, and sixth seal sections 264, 266, 268, 270, 272, 274 with the same ring portion 262 also reduces assembly time and reduces material costs.

The first, second, third, fourth, fifth, and sixth seal sections 264, 266, 268, 270, 272, 274 of the seal assembly 260 are identical to each other in structure and function, and will be described in detail with reference to first seal section 264 shown in FIG. 12. The first seal section 264 includes a wing-shaped body secured to the support member 262 by a connector portion 264a. The connector portion 264a may include a single living hinge, as shown, or a plurality of living hinges (FIG. 4), or may be otherwise configured to permit folding of the first seal section 264 relative to the support member 262. The first seal section 264 and the support member 262 may define a notch 263a on either side of the connector portion 264a to facilitate folding of the first seal section 264 relative to the support member 262. The notches 263a may include a triangular configuration, as shown, or may be otherwise configured to facilitate folding of the first seal section 264 relative to the support member 262.

Inner edge 264b of the first seal section 264 of the seal assembly 260 may define a V-shape, as shown, or may extend straight across. In embodiments, the V-shape defines an angle “α” from about one-hundred eighty degrees (180°) to about two-hundred seventy-five degrees (275°). The V-shape of the inner edge 264b facilitates reception of a surgical instrument (not shown) through the seal assembly 260. Each inner edge 264b may be tapered to further facilitate reception of the surgical instrument through the seal assembly 260 in a sealed manner.

The first seal section 264 defines a plurality of openings 265a adjacent the connector portion 264a between the first seal section 264 and the support member 262, and an opening 265b along each lateral edge 264c of the first seal section 264 adjacent the inner edge 264b of the first seal section 264. The lateral edges 264c of the first seal section 264 each defines a semi-circular cutout 265c between each of the openings 265b and the plurality of openings 265a. The cutouts 265c reduce the amount of material forming the first seal section 264. The cutouts 265c may allow for additional flexing of the first seal section 264.

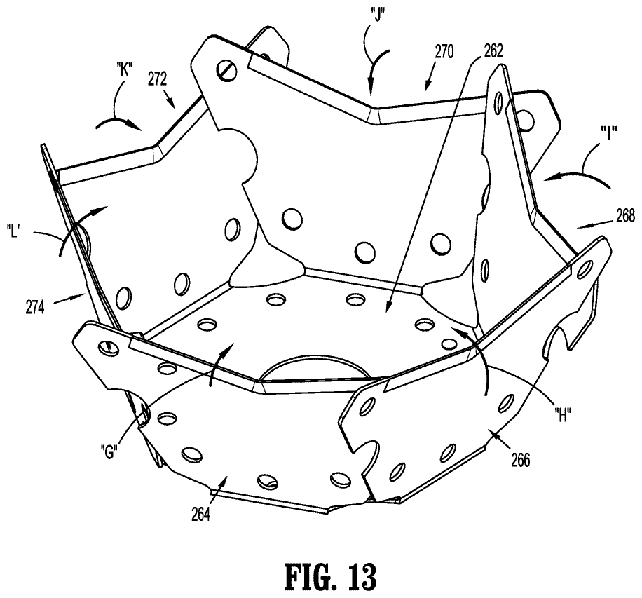

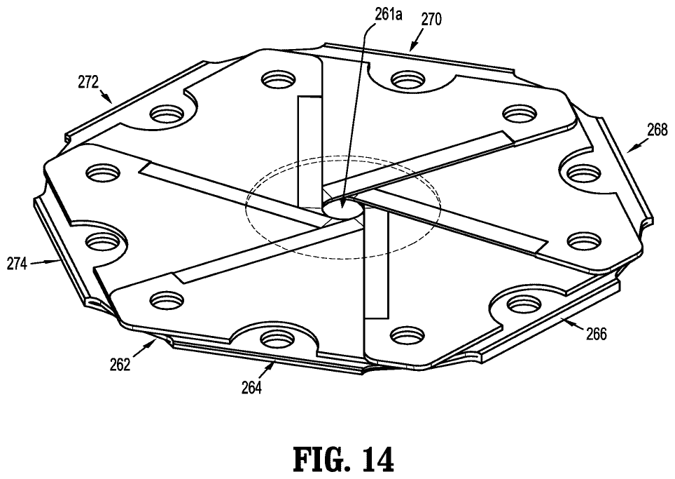

FIG. 13 illustrates the method of folding the seal assembly 260. Each of the first, second, third, fourth, fifth, and sixth seal sections 264, 266, 268, 270, 272, 274 is folded relative to the support member 262, as indicated by arrows “G”, “H”, “I”, “J”, “K”, and “L” respectively. As shown, the first, second, third, fourth, fifth, and sixth seal sections 264, 266, 268, 270, 272, 274 are folded simultaneously, with adjacent seal sections overlapping one another. More particularly, the second seal section 266, overlaps the first seal section 264, the third seal section 268 overlaps the second seal section 266, and so on until the first seal section 264 overlaps the sixth seal section 274. This overlapping or interweaving pattern increases the integrity of the seal assembly 260, i.e., reduces the likelihood of the seal assembly 260 leaking when a surgical instrument is received therethrough. As noted above, alternatively, the seal sections may be folded in any manner, including, with opposed seal sections folded together.

In some aspects of the disclosure, and as shown, the plurality of openings 265a and the openings 265b of the first seal section 264 are arranged such that, when the seal assembly 260 is in the folded condition (FIG. 14), each opening of the plurality of openings 263 in the support member 262 is aligned with an opening of the plurality of openings 265a of the first seal section 264, with two openings of each of the adjacent second and sixth seal sections 266 274, and with one opening of each of the third and fifth seal sections 268, 272. This arrangement ensures the integrity of the seal assembly 260, and more particularly, ensures the positioning of the first, second, third, fifth, and sixth seal sections 264, 266, 268, 2172, 274 relative to each other and the support member 262.

FIG. 14 illustrates the seal assembly 260 in the folded condition. In the folded condition, the first, second, third, fourth, fifth, and sixth seal sections 264, 266, 268, 270, 272, 274 of the seal assembly 260 define an opening 261a configured to receive a surgical instrument (not shown) inserted through the valve assembly 120 in a sealed manner. The first, second, third, fourth, fifth, and sixth seal sections 264, 266, 268, 270, 272, 274 form a non-continuous or virtual seal circumference to reduce tearing during insertion, manipulation, and/or withdrawal of a surgical instrument (not shown) through the valve assembly 120 (FIG. 3).

When the seal assembly 260 is in the folded configuration, the first, second, third, fourth, fifth, and sixth seal sections 264, 266, 268, 270, 272, 274 are configured to partially overlap two adjacent seal sections in each of the clockwise and the counter-clockwise directions. More particularly, the first seal section 264 is configured to partially overlap, the sixth and fifth seal sections 274, 272 in the clockwise direction and the second and third seal section 266, 268 in the counter-clockwise direction, the second seal section 264 is configured to partially overlap the first and sixth seal sections 264, 274 in the clockwise direction and the third and fourth seal sections 264, 268 in the counter-clockwise direction, the third seal section 266 is configured to partially overlap the second and first seal sections 266, 264 in the clockwise direction and the fourth and fifth seal section 270, 272 in the counter-clockwise direction, the fourth seal section 270 is configured to partially overlap the third and second seal sections 268, 266 in the clockwise direction and the fifth and sixth seal sections 272, 274 in the counter-clockwise direction, the fifth seal section 272 is configured to partially overlap the fourth and third seal sections 270, 268 in the clockwise direction and the sixth and first seal sections 274, 264 in the counter-clockwise direction, and the sixth seal section 274 is configured to partially overlap the fifth and fourth seal sections 272, 270 in the clockwise direction and the first and second seal sections 264, 266 in the counter clockwise direction.

In some aspects of the disclosure, and as shown, the first, second, third, fourth, fifth, and sixth seal sections 264, 266, 268, 270, 272, 274 are folded such that the first, second, third, fourth, fifth, and sixth seal sections 264, 266, 268, 270, 272, 274 overlap in a sequential pattern in counter-clockwise direction. Alternatively, the first, second, third, fourth, fifth, and sixth seal sections 264, 266, 268, 270, 272, 274 may folded to overlap in a sequential clockwise direction. It is envisioned that the first, second, third, fourth, fifth, and sixth seal sections 264, 266, 268, 270, 272, 274 may instead be folded in opposed pairs, i.e., first and fourth seal sections 264, 270 folded together, second and fifth seal sections, 266, 272 folded together, and third and sixth seal sections 268, 274 folded together, or in any other suitable manner.

While various embodiments of the present disclosure have been shown and described herein, it will be obvious to those skilled in the art that these embodiments are provided by way of example only. Numerous variations, changes, and substitutions will now occur to those skilled in the art without departing from the present disclosure. Accordingly, it is intended that the present disclosure be limited only by the spirit and scope of the appended claims.

1-20. (canceled)

21. A seal assembly for use with an access assembly configured to receive a surgical instrument, the seal assembly comprising:
a support member; and
a plurality of seal sections coupled with the support member, each seal section of the plurality of seal sections including:
a body portion having a first lateral edge and a second lateral edge, and an inner edge extending between the first lateral edge and the second lateral edge;
wherein the inner edge has a V-shape defining an angle of between about 180 degrees and about 270 degrees,
wherein the first lateral edge and the second lateral edge each include at least one cutout arranged therein.

22. The seal assembly of claim 21, wherein the plurality of seal sections includes six seal sections coupled with the support member.

23. The seal assembly of claim 21, wherein each seal section of the plurality of seal sections is coupled with the support member by a living hinge such that the plurality of seal sections are configured to fold onto the support member in a folded configuration.

24. The seal assembly of claim 21, wherein each seal section of the plurality of seal sections overlaps an adjacent seal section of the plurality of seal sections in a clockwise direction.

25. The seal assembly of claim 24, wherein each seal section of the plurality of seal sections overlaps an adjacent seal section of the plurality of seal sections in a counter-clockwise direction.

26. The seal assembly of claim 25, wherein when the seal assembly is in a folded configuration, the plurality of seal sections and the support member form a non-continuous seal circumference.

27. The seal assembly of claim 21, wherein the support member includes a ring portion having a hexagonal shape and a seal portion.

28. A seal assembly for use with an access assembly configured to receive a surgical instrument, the seal assembly comprising:
a support member; and
a plurality of seal sections coupled with the support member, the plurality of seal sections having a folded configuration defined by each seal section being folded onto the support member, each seal section of the plurality of seal sections comprising:
a body portion having a connector coupled to the support member, the body portion having a first lateral edge opposite a second lateral edge and an inner edge extending between the first lateral edge and the second lateral edge, the inner edge defining a V-shape characterized by an angle,
wherein the V-shape of the inner edge facilitates sealed reception of the surgical instrument through the seal assembly into the access assembly.

29. The seal assembly of claim 28, wherein the support member has a central opening configured for receiving the surgical instrument.

30. The seal assembly of claim 28, wherein the first lateral edge and the second lateral edge of each seal section include at least one cutout, and wherein the at least one cutout facilitates flex in each seal section.

31. The seal assembly of claim 30, wherein the at least one cutout of the first lateral edge and the second lateral edge define a semicircular shape.

32. The seal assembly of claim 28, wherein the angle characterizing the inner edge of each seal section is between about 180 degrees and about 270 degrees.

33. The seal assembly of claim 28, wherein the plurality of seal sections includes six seal sections coupled with the support member.

34. The seal assembly of claim 28, wherein the seal assembly further includes a ring portion defining a hexagonal shape and a seal portion having a plurality of openings arranged circumferentially around the seal portion.

35. The seal assembly of claim 34, wherein each seal section of the plurality of seal sections is connected to the ring portion by a living hinge.

36. The seal assembly of claim 34, wherein the surgical access assembly further includes a retainer assembly, the retainer assembly including an upper retainer member, a lower retainer member, and a plurality of pins extending from one of the upper retainer member or the lower retainer member, wherein each pin of the plurality of pins is received through an opening in three seal sections of the plurality of seal sections and through a first opening of the plurality of openings of the seal portion.

37. A seal assembly for use with an access assembly configured to receive a surgical instrument, the seal assembly comprising:
a support member having a central opening and a plurality of openings circumferentially arranged around the support member; and
a plurality of seal sections rotatability secured with the support member, each seal section of the plurality of seal sections including:
a body having a connector coupled with the support member, and
a first lateral edge and a second lateral edge extending from the connector, the first lateral edge and the second lateral edge each having at least one cutout defined therein;
wherein the plurality of seal sections are arranged in a folded configuration defined by each seal section being folded onto the support member and at least partially overlapped with an adjacent seal section.

38. The seal assembly of claim 37, wherein the folded configuration is further defined by the plurality of seal sections forming a non-continuous seal circumference configured to reduce tearing during one or more of insertion, manipulation, or withdrawal of the surgical instrument.

39. The seal assembly of claim 37, wherein each seal section of the plurality of seal sections overlaps an adjacent seal section of the plurality of seal sections in a clockwise direction.

40. The seal assembly of claim 39, wherein each seal section of the plurality of seal sections overlaps an adjacent seal section of the plurality of seal sections in a counter-clockwise direction.
